(19) United States
(12) Patent Application Publication
(10) Pub. No.: US 2017/0360850 A1
(43) Pub. Date: Dec. 21, 2017

(54) PROBIOTIC ORGANISMS FOR DIAGNOSIS, MONITORING, AND TREATMENT OF INFLAMMATORY BOWEL DISEASE

(71) Applicants: Massachusetts Institute of Technology, Cambridge, MA (US); Synlogic, Inc., Cambridge, MA (US)

(72) Inventors: Timothy Kuan-Ta Lu, Cambridge, MA (US); Jacob Rosenblum Rubens, Cambridge, MA (US); Isaak Elis Mueller, Cambridge, MA (US); Gianluca Selvaggio, Rimini (IT); Paul Miller, Salem, CT (US); Dean Falb, Sherborn, MA (US); Vincent Isabella, Cambridge, MA (US); Jonathan Kotula, Somerville, MA (US)

(73) Assignees: Massachusetts Institute of Technology, Cambridge, MA (US); Synlogic, Inc., Cambridge, MA (US)

(21) Appl. No.: 15/535,831

(22) PCT Filed: Dec. 22, 2015
(86) PCT No.: PCT/US2015/067435

§ 371 Date: Jun. 14, 2017

Related U.S. Application Data
(60) Provisional application No. 62/095,415, filed on Dec. 22, 2014.

(51) Int. Cl. A61K 35/741; C07K 16/24; C07K 14/54; A61K 39/395; A61K 38/20; A61K 36/064; A61K 31/122; C12N 15/70; A61K 9/00

(52) U.S. Cl. CPC A61K 35/741; C12N 15/70; C07K 16/241; C07K 14/5428; A61K 9/0053; A61K 38/2066; A61K 36/064; A61K 31/122; A61K 39/3955

(57) ABSTRACT

Aspects of the present disclosure relate to genetically engineered organisms useful for the diagnosis and treatment of inflammatory bowel disease.

### RELATED APPLICATION

This application claims the benefit under 35 U.S.C. §119(e) of U.S. provisional application No. 62/095,415, filed Dec. 22, 2014, which is incorporated by reference herein in its entirety.

### FEDERALLY SPONSORED RESEARCH

This invention was made with Government support under HDTRA1-14-1-0007 awarded by the Defense Threat Reduction Agency and under Grant No. N00014-11-1-0725 awarded by the Office of Naval Research. The Government has certain rights in the invention.

### FIELD

Some aspects of the present disclosure relate to the field of biosynthetic engineering. Some aspects of the present disclosure relate to the methods and compositions for the diagnosis and treatment of inflammatory bowel disease.

### BACKGROUND

Inflammatory bowel disease (IBD) is a chronic inflammatory disease encompassing Crohn's disease and ulcerative colitis. To date, the cause for IBD is not known and there is no cure. Moreover, current IBD therapies involve high, general dosing of anti-inflammatory agents that can lead to severe side effects, such as immunodeficiency. It is also challenging to detect early disease flares in a non-invasive fashion, thus making it difficult to treat the disease in patients before symptoms become severe. Thus, new technologies are needed to improve the study of IBD in animal models, to enable early detection of disease flares, and to achieve targeted delivery of anti-inflammatory therapies.

### SUMMARY

In some aspects the disclosure relates to a recombinant probiotic cell comprising a sensor circuit, comprising: (a) a first promoter operably linked to a nucleic acid encoding a regulatory protein responsive to an input signal; (b) a second promoter responsive to the regulatory protein and operably linked to a nucleic acid encoding a first output protein, wherein activity of the second promoter is altered when bound by the regulatory protein; (c) an output molecule operably linked to a third promoter, wherein the output molecule or the third promoter is flanked by a first set of regulatory sequences, wherein the first set of regulatory sequences interacts with the first output protein to unlink the output molecule from the third promoter; (d) a fourth promoter responsive to the regulatory protein and operably linked to a nucleic acid encoding a second output protein, wherein activity of the fourth promoter is altered when bound by the regulatory protein; and, optionally, (e) a second output molecule flanked by a second set of regulatory sequences, wherein the second set of regulatory sequences interacts with the second output protein to operably link the second output molecule to a fifth promoter.

In some embodiments, the promoter of (a) is a constitutively-active promoter. In some embodiments, the regulatory protein is selected from the group consisting of oxyR, NorR, and NsrR. In some embodiments, the input signal is hydrogen peroxide (H₂O₂). In some embodiments, the input signal is nitric oxide (NO). In some embodiments, the input signal is an inflammatory cytokine, optionally IL-6, IL-18, or TNFα. In some embodiments, the input molecule is a molecule produced by neutrophils, such as calprotectin or lactoferrin. In some embodiments, the input signal is blood.

In some embodiments, the promoter of (b) and/or (d) comprises a modification that alters the binding affinity of a transcription factor or RNA polymerase for the promoter of (b) and/or (d), relative to a similar unmodified promoter. In some embodiments, the modification is a nucleic acid mutation. In some embodiments, (a), (b) and (c) as described above are on a vector. In some embodiments, (a), (b), (c) and (d) as described above are on a vector. In some embodiments, (a) and (b) are on a single vector. In some embodiments, (a), (b) and (d) are on a single vector. In some embodiments, (c) and/or (e) is on a bacterial artificial chromosome (BAC).

In some embodiments, (b) and/or (d) further comprises a sequence element that regulates production of the first output protein and is located between the second promoter and the nucleic acid encoding the first output protein. In some embodiments, the sequence element regulates transcription or translation of the output protein. In some embodiments, the sequence element is a ribosomal binding site. In some embodiments, the sequence element is a modified ribosomal binding site comprising a modification that alters the binding affinity of a ribosome for the modified ribosomal binding site, relative to a similar unmodified ribosomal binding site.

In some embodiments, the promoter of (b) and/or (d) is a promoter selected from the group consisting of oxyR, katGp oxySp, ahpSp, HemHp, ahpCp2, dsbGp, uofp, dpsp, grxAp, ybjCp, hcpp, ychFp, sufAp, flup, mntHp, trxCp, gorp, yhjAp, oxyRp, gntPp, uxuAp, fhuFp, katGp, nir, hcp, nrfA, nasD, ytfE, yeaR, nnrS and norV that comprises a modification that alters the binding affinity of a transcription factor or RNA polymerase for a promoter selected from the group consisting of oxyR, katGp oxySp, ahpSp, HemHp, ahpCp2, dsbGp, uofp, dpsp, grxAp, ybjCp, hcpp, ychFp, sufAp, flup, mntHp, trxCp, gorp, yhjAp, oxyRp, gntPp, uxuAp, fhuFp, katGp, nir, hcp, nrfA, nasD, ytfE, yeaR, nnrS and norV of (b), relative to a similar unmodified promoter. In some embodiments, the promoter of (b) and/or (d) is a promoter selected from the group consisting of oxyR, katGp oxySp, ahpSp, HemHp, ahpCp2, dsbGp, uofp, dpsp, grxAp, ybjCp, hcpp, ychFp, sufAp, flup, mntHp, trxCp, gorp, yhjAp, oxyRp, gntPp, uxuAp, fhuFp, katGp, nir, hcp, nrfA, nasD, ytfE, yeaR, nnrS and norV that is a naturally occurring promoter.

In some embodiments, the first output protein of (b) is a recombinase and the first set of regulatory sequences of (c) is recombinase recognition sites. In some embodiments, the second output protein of (d) is a recombinase and the second set of regulatory sequences of (e) is recombinase recognition sites. In some embodiments, the first output molecule and/or the second output molecule is detectable. In some embodiments, the first output molecule and/or the second output molecule is detectable by PCR, DNA sequencing or microscopy, optionally fluorescent microscopy.

In some embodiments, the first output molecule and/or the second output molecule is a therapeutic molecule. In some embodiments, the first output molecule and the second output molecule are the same molecule. In some embodiments, the therapeutic molecule is an anti-inflammatory molecule. In some embodiments, the anti-inflammatory molecule is a cytokine, optionally IL-10. In some embodiments, the anti-inflammatory molecule is curcumin.

In some embodiments, the cell is a bacterial cell or a fungal cell. In some embodiments, the bacterial cell is an E. coli cell, optionally an E. coli Nissle 1917 cell. In some embodiments, the fungal cell is a yeast cell, optionally, a Saccharomyces boulardii cell.

In some aspects, the disclosure relates to a method of treating an inflammatory bowel disease in a subject in need thereof, the method comprising administering to a subject in need thereof a probiotic cell as described herein.

In some embodiments of the method, the inflammatory bowel disease is Crohn's disease or ulcerative colitis.

In some embodiments of the method, the sensor circuit is a biological analog signal processing circuit. In some embodiments of the method, the input signal is hydrogen peroxide (H₂O₂). In some embodiments of the method, the input signal is nitric oxide (NO). In some embodiments of the method, the input molecule is an inflammatory cytokine, optionally IL-6, IL-18, or TNFα. In some embodiments, the input molecule is a molecule produced by neutrophils, such as calprotectin or lactoferrin. In some embodiments, the input signal is blood.

In some embodiments of the method, the first and/or second output molecule is detectable. In some embodiments of the method, the first and/or second output molecule is detectable by PCR, DNA sequencing or microscopy, optionally fluorescent microscopy. In some embodiments of the method, the output molecule is detectable via colorimetric observations.

In some embodiments of the method, the first and/or second output molecule is a therapeutic molecule. In some embodiments of the method, the therapeutic molecule is an anti-inflammatory molecule. In some embodiments of the method, the anti-inflammatory molecule is a cytokine, optionally IL-10. In some embodiments of the method, the anti-inflammatory molecule is curcumin. In some embodiments of the method, the anti-inflammatory molecule is an antibody or antibody fragment. In some embodiments of the method, the sensor circuit further comprises a second output molecule. In some embodiments of the method, the second output molecule is the same as the first output molecule.

In some aspects, the disclosure relates to a method of diagnosing an inflammatory bowel disease in a subject, the method comprising: a) administering to a subject a probiotic cell as described herein; b) obtaining a biological sample from the subject of (a); c) detecting the expression of the at least one output molecule in the biological sample; and, d) diagnosing the subject as having an inflammatory bowel disease.

In some embodiments of the method, the probiotic cell colonizes the intestinal tract. In some embodiments of the method, the subject is a mammal. In some embodiments of the method, the subject is a human. In some embodiments of the method, the biological sample is a fecal sample. In some embodiments of the method, the expression of the output molecule is detected in vitro. In some embodiments of the method, the inflammatory bowel disease is Crohn's disease or ulcerative colitis. In some embodiments, the method further comprises administering an agent useful for treatment of IBD to the subject.

In another aspect, recombinant probiotic cells are provided that include a sensor circuit that includes (a) a first promoter operably linked to a nucleic acid encoding a regulatory protein responsive to an input signal; and (b) a second promoter responsive to the regulatory protein and operably linked to a nucleic acid encoding a first output protein, wherein activity of the second promoter is altered when bound by the regulatory protein. In some embodiments, the first promoter is a constitutively-active promoter.

In some embodiments, the regulatory protein is selected from the group consisting of oxyR, NorR, and NsrR. In some embodiments, the input signal is hydrogen peroxide (H₂O₂). In some embodiments, the input signal is nitric oxide (NO). In some embodiments, the input signal is an inflammatory cytokine, optionally IL-6, IL-18, or TNFα.

In some embodiments, the second promoter includes a modification that alters the binding affinity of a transcription factor or RNA polymerase for the second promoter, relative to a similar unmodified promoter. In some embodiments, the modification is a nucleic acid mutation.

In some embodiments, (a) and (b) are on a vector, in some embodiments, on a single vector.

In some embodiments, (b) further includes a sequence element that regulates production of the first output protein and is located between the second promoter and the nucleic acid encoding the first output protein. In some embodiments, the sequence element regulates transcription or translation of the output protein. In some embodiments, the sequence element is a ribosomal binding site. In some embodiments, the sequence element is a modified ribosomal binding site comprising a modification that alters the binding affinity of a ribosome for the modified ribosomal binding site, relative to a similar unmodified ribosomal binding site.

In some embodiments, the promoter of (b) is a promoter selected from the group consisting of oxyR, oxySp, katGp, nir, hcp, nrfA, nasD, ytfE, yeaR, nnrS and norV that comprises a modification that alters the binding affinity of a transcription factor or RNA polymerase for a promoter selected from the group consisting of oxyR, nir, hcp, nrfA, nasD, ytfE, yeaR, nnrS and norV of (b), relative to a similar unmodified promoter. In some embodiments, the promoter of (b) is a promoter selected from the group consisting of oxyR, oxySp, katGp, nir, hcp, nrfA, nasD, ytfE, yeaR, nnrS and norV that is a naturally occurring promoter.

In some embodiments, the first output molecule is a therapeutic molecule. In some embodiments, the therapeutic molecule is an anti-inflammatory molecule. In some embodiments, the anti-inflammatory molecule is a cytokine, optionally IL-10.

In some embodiments, the anti-inflammatory molecule is curcuminoid synthase (CUS) that converts feruloyl-CoA to curcumin. In some embodiments, the recombinant probiotic cell further includes nucleic acids that encode 4-coumarate:CoA ligase and acetyl-CoA carboxylase which nucleic acids optionally are on one or more vectors. In some embodiments, the nucleic acids that encode acetyl-CoA carboxylase are AccBc and DtsR1. In some embodiments, nucleic acids that encode 4-coumarate:CoA ligase and acetyl-CoA carboxylase are operably linked to constitutive promoters.

In some embodiments, the cell is a bacterial cell or a fungal cell. In some embodiments, the bacterial cell is an E. coli cell, optionally an E. coli Nissle 1917 cell. In other embodiments, the fungal cell is a yeast cell, optionally, a Saccharomyces boulardii cell.

In another aspect, methods of treating an inflammatory bowel disease in a subject in need thereof are provided. The methods include administering to a subject in need thereof the foregoing probiotic cell that include a sensor circuit that includes (a) a first promoter operably linked to a nucleic acid encoding a regulatory protein responsive to an input signal; and (b) a second promoter responsive to the regulatory protein and operably linked to a nucleic acid encoding a first output protein, wherein activity of the second promoter is altered when bound by the regulatory protein. In some embodiments, the inflammatory bowel disease is Crohn's disease or ulcerative colitis.

### BRIEF DESCRIPTION OF DRAWINGS

FIGS. 1A-1D provide an overview of engineered probiotics for sensing-and-treating inflammation. FIG. 1A shows probiotic cells (for example, E. coli) pass through the gut and encounter sites of inflammation (squares and “X”). FIG. 1B shows the design of probiotic cells engineered to sense and memorize the presence and concentration of inflammatory markers (dots), thus enabling early detection of inflammation (light shaded cells, right). FIG. 1C shows the design of probiotic cells that constitutively synthesize anti-inflammatory molecules (circles). FIG. 1D shows the design of targeted therapies for IBD via probiotic cells that can sense inflammation (squares, “X”) and respond by locally secreting anti-inflammatory therapies (circles).

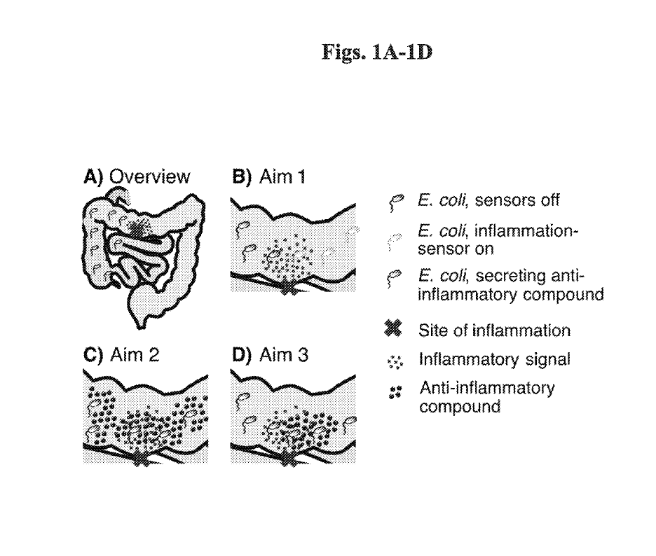

FIGS. 2A-2C show a schematic for engineered inflammation-sensing circuits with integrated memory based on DNA recombinases. FIG. 2A shows a schematic demonstrating the expression of three different DNA recombinases (Rec. 1, 2, 3) is induced at different levels of inflammatory molecules, such as H₂O₂ and NO. FIG. 2B shows that upon expression, each DNA recombinase (R1, R2, R3) shown in FIG. 2A inverts an independent output DNA sequence, thus resulting in permanent expression of a separate output gene contained within the inverted sequence (Output 1, Output 2, Output 3, respectively). These output genes can include detectable reporters, as well as therapeutic proteins or small-molecule biosynthetic genes. The status of these output modules can also be read out via DNA sequencing and PCR. The output modules may also be read out via microscopy or colorimetric observation. FIG. 2C shows a schematic representation of the response of the engineered probiotic cells to an inflammatory marker. Multiple output genes are expressed depending on the level of inflammation that has been encountered. For example, inflammation levels that are between Threshold 1 and Threshold 2 would only induce the Output 1 (left curve), but inflammation levels above Threshold 3 would induce Output 1, Output 2 (middle curve), and Output 3 (right curve) outputs. Thus, permanent readouts of inflammatory conditions encountered by probiotic bacteria can be recorded in DNA.

FIG. 3 shows three different biosensing circuits expressing GFP for detecting H₂O₂ levels were constructed in E. coli using two different promoters (oxySp and katGp) combined with three different ribosome binding sites (0033, 0031, 0029). The resulting circuit designs yield two different input-output transfer functions that have different thresholds and sensitivities for sensing H₂O₂ levels.

FIGS. 4A-4B show curcumin production in E. coli. FIG. 4A shows the production of curcumin from ferulic acid requires expression of four heterologous enzymes. FIG. 4B shows a gene circuit for inflammation-inducible curcumin production. To enable controlled production of curcumin by inflammation-sensing circuits, expression of the curcuminoid synthase (CUS) enzyme needed for the last conversion step (feruloyl-CoA to curcumin) is placed under regulation by inflammation sensors, for example those described in FIGS. 2A-2C.

### DETAILED DESCRIPTION

In some aspects, the disclosure relates to the use of probiotic bacteria as non-invasive sensors of inflammation and producers of localized anti-inflammatory compounds to treat inflammatory bowel disease (IBD). These probiotics can be consumed orally in order to diagnose and treat IBD as they transit through the gut. Furthermore, engineered probiotics can be recovered from stool and interrogated to recover information on the conditions they encountered during their transit through the gut.

Accordingly, in some aspects, the disclosure relates to a recombinant probiotic cell comprising a sensor circuit, comprising: (a) a first promoter operably linked to a nucleic acid encoding a regulatory protein responsive to an input signal; (b) a second promoter responsive to the regulatory protein and operably linked to a nucleic acid encoding a first output protein, wherein activity of the second promoter is altered when bound by the regulatory protein; (c) an output molecule operably linked to a third promoter, wherein the output molecule or the third promoter is flanked by a first set of regulatory sequences, wherein the first set of regulatory sequences interacts with the first output protein to unlink the output molecule from the third promoter; (d) a fourth promoter responsive to the regulatory protein and operably linked to a nucleic acid encoding a second output protein, wherein activity of the fourth promoter is altered when bound by the regulatory protein; and, optionally, (e) a second output molecule flanked by a second set of regulatory sequences, wherein the second set of regulatory sequences interacts with the second output protein to operably link the second output molecule to a fifth promoter.

### Probiotic Cells

Provided herein are probiotic cells comprising sensor circuits. As used herein, the term “probiotic” refers to live micro-organisms which, when administered in adequate amounts, confer a health benefit on the host to which they are administered. For example, E. coli Nissle 1917 bacteria were used to successfully treat an outbreak of shigellosis during World War I. Examples of probiotic organisms include bacteria (Lactobacillus acidophilus, Lactobacillus paracasei, Lactobacillus johnsonii, Lactobacillus plantarum, Lactobacillus reuteri ATCC 55730, Bifidobacterium longum, Bacillus coagulans, and Escherichia coli Nissle 1917 (EcN)) and fungi (e.g. Saccharomyces cerevisiae, Saccharomyces boulardii, Saccharomyces pastoriamus, Saccharomyces batanus).

In some embodiments, probiotic cells of the present disclosure are anaerobic bacterial cells (e.g., cells that do not require oxygen for growth). Anaerobic bacterial cells include facultative anaerobic cells such as, for example, Escherichia coli and Lactobacillus sp. In some embodiments, the probiotic cell comprising a sensor circuit is an E. coli cell. In some embodiments, the E. coli cell is an E. coli Nissle 1917 (EcN) cell. In some embodiments, probiotic cell of the present disclosure is a yeast cell. In some embodiments, the yeast cell is a Saccharomyces boulardii cell.

As used herein, the term “recombinant cell” refers to a cell that has been engineered through genetic recombination to comprise nucleic acids that are not naturally be present in said cell. A recombinant cell contains an exogenous nucleic acid or a nucleic acid that does not occur in nature (e.g., sensor circuit of the present disclosure). In some embodiments, a recombinant cell contains an exogenous independently replicating nucleic acid (e.g., components of analog signal processing circuits present on an episomal vector). In some embodiments, a recombinant cell is produced by introducing a foreign or exogenous nucleic acid into a cell. Thus, provided herein are methods of introducing a circuit into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88).

In some embodiments, a cell is modified to overexpress an endogenous protein of interest (e.g., via introducing or modifying a promoter or other regulatory element near the endogenous gene that encodes the protein of interest to increase its expression level). In some embodiments, a cell is modified by mutagenesis. In some embodiments, a cell is modified by introducing an engineered nucleic acid into the cell in order to produce a genetic change of interest (e.g., via insertion or homologous recombination). In some embodiments, a cell contains a gene deletion.

Analog signal processing circuits of the present disclosure may be transiently expressed or stably expressed. “Transient cell expression” refers to expression by a cell of a nucleic acid that is not integrated into the nuclear genome of the cell. By comparison, “stable cell expression” refers to expression by a cell of a nucleic acid that remains in the nuclear genome of the cell and its daughter cells. Typically, to achieve stable cell expression, a cell is co-transfected with a marker gene and an exogenous nucleic acid (e.g., an analog signal processing circuit or component thereof) that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Examples of marker genes and selection agents for use in accordance with the present disclosure include, without limitation, dihydrofolate reductase with methotrexate, glutamine synthetase with methionine sulphoximine, hygromycin phosphotransferase with hygromycin, puromycin N-acetyltransferase with puromycin, and neomycin phosphotransferase with Geneticin, also known as G418. Other marker genes/selection agents are contemplated herein.

### Biological Sensor Circuits

In some aspects, the disclosure relates to recombinant probiotic cells comprising a sensor circuit. As used herein, a “sensor circuit” refers to a genetic circuit used to detect a biological signal, for example an inflammatory marker. Biological signals are often present in dynamic concentration gradients and in some cases it is desirable to convert a gradient of input signal into discreet expression of a molecule or molecules (e.g. via the application of analog to digital logic). Therefore, in some embodiments, the sensor circuit is a biological analog signal processing circuit. For a further description of biological analog signal processing circuits, see U.S. Ser. No. 62/095,318 (Attorney Docket No. M0656.70347US00), titled “Analog to Digital Computations in Biological Systems” and filed of even date, herein incorporated by reference in its entirety.

Analog signal processing circuits of the present disclosure comprise promoters responsive to an input signal and operably linked to a nucleic acid encoding an output molecule. A “promoter” is a control region of a nucleic acid at which initiation and rate of transcription of the remainder of a nucleic acid are controlled. A promoter may also contain sub-regions at which regulatory proteins and molecules, such as transcription factors, bind. Promoters of the present disclosure may be constitutive, inducible, activatable, repressible, tissue-specific or any combination thereof. A promoter drives expression or drives transcription of the nucleic acid that it regulates. A promoter is considered to be “operably linked” when it is in a correct functional location and orientation in relation to the nucleic acid it regulates to control (“drive”) transcriptional initiation and/or expression of that nucleic acid.

A promoter is considered “responsive” to an input signal if the input signal modulates the function of the promoter, indirectly or directly. In some embodiments, an input signal may positively modulate a promoter such that the promoter activates, or increases (e.g., by a certain percentage or degree), transcription of a nucleic acid to which it is operably linked. In some embodiments, by contrast, an input signal may negatively modulate a promoter such that the promoter is prevented from activating or inhibits, or decreases, transcription of a nucleic acid to which it is operably linked. An input signal may modulate the function of the promoter directly by binding to the promoter or by acting on the promoter without an intermediate signal. For example, the oxyR protein modulates the oxyR promoter by binding to a region of the oxyR promoter. Thus, the oxyR protein is herein considered an input signal that directly modulates the oxyR promoter. By contrast, an input signal is considered to modulate the function of a promoter indirectly if the input signal modulates the promoter via an intermediate signal. For example, hydrogen peroxide (H₂O₂) modulates (e.g., activates) the oxyR protein, which, in turn, modulates (e.g., activates) the oxyR promoter. Thus, H₂O₂ is herein considered an input signal that indirectly modulates the oxyR promoter.

An “input signal” refers to any chemical (e.g., small molecule) or non-chemical (e.g., light or heat) signal in a cell, or to which the cell is exposed, that modulates, directly or indirectly, a component (e.g., a promoter) of an analog signal processing circuit. In some embodiments, an input signal is a biomolecule that modulates the function of a promoter (referred to as direct modulation), or is a signal that modulates a biomolecule, which then modulates the function of the promoter (referred to as indirect modulation). A “biomolecule” is any molecule that is produced in a live cell, e.g., endogenously or via recombinant-based expression. H₂O₂ and Nitric oxide (NO) are considered input signals that indirectly modulate the oxyR promoter and nir, hcp, nrfA, nasD, ytfE, yeaR, nnrS and norV promoters, respectively, and, in turn, expression of output molecules. Likewise, the oxyR and NorR or NsrR proteins are themselves considered input signals because they directly modulate transcription of output molecules by binding to oxyR promoter(s) (for example oxyR, katGp oxySp, ahpSp, HemHp, ahpCp2, dsbGp, uofp, dpsp, grxAp, ybjCp, hcpp, ychFp, sufAp, flup, mntHp, trxCp, gorp, yhjAp, oxyRp, gntPp, uxuAp, fhuFp, katGp) or nir, or hcp, or nrfA, or nasD, or ytfE, or yeaR, or nnrS or norV, respectively. In some embodiments, an input signal may be endogenous to a cell or a normally exogenous condition, compound or protein that contacts a promoter of an analog signal processing circuit in such a way as to be active in modulating (e.g., inducing or repressing) transcriptional activity from a promoter responsive to the input signal (e.g., an inducible promoter). In some embodiments, an input signal is constitutively expressed in a cell. In some embodiments, the input signal is oxyR protein. In some embodiments, the input signal is NorR or NsrR protein.

In some aspects, the disclosure relates to sensor circuits responsive to inflammatory marker input signals. As used herein, the term “inflammatory marker” relates to any chemical or biological indicator of an inflammatory immune response. Examples of inflammatory markers include but are not limited to IL-1, IL-6, IL-18, TNF-α, IFN-γ, H₂O₂, NO, blood, Calprotectin, Lactoferrin, other molecules associated with neutrophil invasion into the gut lumen. In particular, inflammatory markers associated with inflammatory bowel disease (IBD) are contemplated as input signals. As used herein, “inflammatory bowel disease” refers to a heterogeneous group of chronic inflammatory disorders of the gastrointestinal tract that includes Crohn's disease (CD) and ulcerative colitis (UC). In some embodiments, the input signal is H₂O₂. In some embodiments, input signal is NO. In some embodiments, the input molecule is selected from the group consisting of IL-6, IL-18, or TNFα. In some embodiments, the input molecule is a molecule produced by neutrophils, such as calprotectin or lactoferrin. In some embodiments, the input signal is blood. Combinations of input signals are also contemplated, for example a recombinant probiotic cell comprising a sensor circuit responsive to two or more of the input signals selected from the group consisting of H₂O₂, NO, IL-6, IL-18, TNFα, Blood, Calprotectin, Lactoferrin.

In some embodiments, the sensor circuit comprises a promoter that is operably linked to a nucleic acid encoding an output molecule (e.g., a recombinase or a detectable protein). In some embodiments, output promoters are responsive to a regulatory protein, such as, for example, a transcription factor. In some embodiments, output promoters are modified (e.g., mutated) such that the affinity of the promoter for a particular regulatory protein is altered (e.g., reduced), relative to the affinity of the unmodified promoter for that same regulatory protein. Alternatively, output promoters are naturally occurring promoters that bind the same transcription factor with different affinities. For example, oxySp and KatGp are two naturally-occurring promoters that bind the oxyR protein with different affinities.

Recombinases are enzymes that mediate site-specific recombination by binding to nucleic acids via conserved recognition sites and mediating at least one of the following forms of DNA rearrangement: integration, excision/resolution and/or inversion. Recombinases are generally classified into two families of proteins, tyrosine recombinases (YR) and serine recombinases (SR). However, recombinases may also be classified according to their directionality (i.e. bidirectional or unidirectional).

Unidirectional recombinases bind to non-identical recognition sites and therefore mediate irreversible recombination. Examples of unidirectional recombinase recognition sites include attB, attP, attL, attR, pseudo attB, and pseudo attP. In some embodiments, the circuits described herein comprise unidirectional recombinases. Examples of unidirectional recombinases include but are not limited to BxbI, PhiC31, TP901, HK022, HP1, R4, Int1, Int2, Int3, Int4, Int5, Int6, Int1, Int8, Int9, Int10, Int11, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34. Further unidirectional recombinases may be identified using the methods disclosed in Yang et al., Nature Methods, October 2014; 11(12), pp. 1261-1266, herein incorporated by reference in its entirety.

In some embodiments of the circuits described herein, the circuit(s) comprise at least one unidirectional recombinase, wherein the recognition sites flanking a nucleic acid sequence are operable with the at least one unidirectional recombinase. In some embodiments, the circuit(s) comprise two or more unidirectional recombinases.

Also contemplated herein are biological signal processing circuits that are reversible. Reversible biological signal processing circuits allow the expression of an output molecule to be turned on and off, for example via the use of a “reset switch” or a second circuit that reverses the activity of an activated regulatory protein. In some embodiments, the biological signal processing circuit comprises at least one bidirectional recombinase. Bidirectional recombinases bind to identical recognition sites and therefore mediate reversible recombination. Examples of bidirectional recombinases include, but are not limited to, Cre, FLP, R, IntA, Tn3 resolvase, Hin invertase and Gin invertase. In some embodiments, the output molecule is flanked by at least one bidirectional recombinase recognition site. In some embodiments, the bidirectional recombinase recognition sites flanking an output molecule are the same. In some embodiments, the bidirectional recombinase recognition sites flanking an output molecule are different. Non-limiting examples of identical recognition sites for bidirectional recombinases include loxP, FRT and RS recognition sites. Non-limiting examples of identical recognition sites for bidirectional recombinases include loxP, FRT and RS recognition sites. It should also be noted that bidirectional recombinases can be engineered or modified to behave as unidirectional recombinases. For example, tyrosine recombinases, such as CRE can be utilized in combination with two different recombinase recognition sites (e.g. lox66 and lox71).

In some embodiments, a reversible biological analog signal processing circuit comprises a reset switch. In some embodiments, the reset switch comprises at least one recombinase directionality factor (RDF) that alters the action of a recombinase. Recombinase directionality factors are known in the art and are described, for example in Bonnet et al. PNAS 109(23), pp. 8884-9, 2012 (herein incorporated by reference in its entirety).

In some embodiments, the biological analog signal processing circuits described herein comprise bacterial recombinases. A non-limiting examples of bacterial recombinases include the FimE, FimB, FimA and HbiF. HbiF is a recombinase that reverses recombination sites that have been inverted by Fim recombinases. Bacterial recombinases recognize inverted repeat sequences, termed inverted repeat right (IRR) and inverted repeat left (IRL). In some embodiments, biological analog signal processing circuits comprising bacterial recombinases further comprise a bacterial recombinase regulator. A non-limiting example of a bacterial recombinase regulator is PapB, which inhibits FimB activity.

Sensor circuits, and components thereof, of the disclosure can be “tuned” by promoter modification such that the affinity of a promoter for a regulatory protein differs relative to the affinity of another promoter for the same regulatory protein. Further tuning of analog signal processing circuits is contemplated herein. For example, a “regulatory sequence” may be included in a circuit to further regulate transcription, translation or degradation of an output molecule or regulatory protein. Examples of regulatory sequences as provided herein include, without limitation, ribosomal binding sites, riboswitches, ribozymes, guide RNA binding sites, microRNA binding sites, toe-hold switches, cis-repressing RNAs, siRNA binding sites, protease target sites, recombinase recognition sites and transcriptional terminator sites.

In some aspects, the disclosure relates to a biological analog signal processing circuit comprising regulatory sequences. In some embodiments, the regulatory sequences are recombinase recognition sites. In some embodiments, the recombination recognition sites recognize a recombinase selected from the group consisting of BxbI, PhiC31, TP901, BxbI, PhiC31, TP901, HK022, HP1, R4, Int1, Int2, Int3, Int4, Int5, Int6, Int1, Int8, Int9, Int10, Int11, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34. In some embodiments, the biological analog signal processing circuit comprises two or more different regulatory sequences. In some embodiments, the regulatory sequences regulate the transcription and/or translation of an output molecule. In some embodiments, the regulatory sequences regulate the operable linkage of a promoter to a nucleic acid sequence encoding an output protein. In some embodiments, a first set of regulatory sequences regulates the transcription and/or translation of an output molecule and a second set of regulatory sequences regulates the operable linkage of a promoter to a nucleic acid sequence encoding an output protein.

Tuning may also be achieved by modifying (e.g., mutating) a ribosomal binding site (RBS) located between a promoter and a nucleic acid to which it is operably linked. In some embodiments, the biological circuits described herein comprise RBS that have different translation efficiencies. In some embodiments, the RBSs are naturally occurring RBSs. In some embodiments, the RBSs are modified RBSs. In some embodiments, modified RBS have different translation efficiencies as a result of at least one modification relative to a wild-type (unmodified) version of the same RBS.

Tuning also can be achieved by changing the affinity of RNA polymerase for the promoter, and thus the strength of the promoter. For example, one or more mutations are made in the −10 region of the promoter. By changing the promoter strength (and thus transcription rate of the recombinase), digital switches are obtained (with regards to an input, such as H2O2) at different concentrations.

Tuning of an analog signal processing circuit may also be achieved, for example, by controlling the level of nucleic acid expression of particular components of the circuit. This control can be achieved, for example, by controlling copy number of the nucleic acids (e.g., using low, medium and/or high copy plasmids, and/or constitutively-active promoters).

It should be understood that the “tunability” of analog signal processing circuits of the present disclosure is achieved, in some embodiments, by combining two or more tuning mechanisms as provided herein. For example, in some embodiments, analog signal processing circuits comprise at least one modified promoter (with reduced or increased affinity for a regulatory protein) and a ribosome binding site (RBS). In some embodiments, analog signal processing circuits comprise a modified promoter and at least one modified ribosomal binding site. In some embodiments, analog signal processing circuits comprise a modified ribosomal binding site and regulatory sequence. Other configurations are contemplated herein.

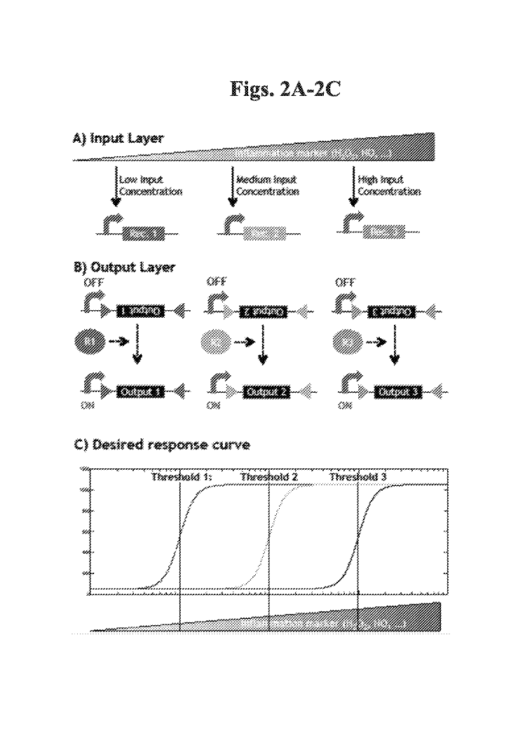

Sensor circuits of the present disclosure, in some embodiments, generate a response in the form of an output molecule. An “output molecule” refers to any detectable molecule under the control of (e.g., produced in response to) an input signal. For example, as shown in FIG. 2, Output 1, Output 2 and Output 3 are output molecules produced in response to activation of a promoter driving expression of a recombinase gene (Rec. 1, Rec. 2 and Rec. 3) by an inflammatory marker. The expression level of an output molecule, in some embodiments, depends on the affinity of a promoter for a particular regulatory protein. For example, the expression level of an output protein under the control of a modified promoter having reduced affinity for a regulatory protein may be less than the expression level of an output molecule under the control of the unmodified promoter. Likewise, the expression level of an output molecule under the control of a modified promoter having reduced affinity for a regulatory protein may be less than the expression level of an output molecule under the control of a modified promoter having an even greater reduction in its affinity for the same regulatory protein.

Examples of output molecules include, without limitation, proteins and nucleic acids. In some embodiments, output molecules are detectable. Detectable output molecules are useful for the formation of DNA memory. For example, an input signal can activate expression of a recombinase, which irreversibly flips a specific stretch of DNA, thus creating a stable memory of events that can be read out via reporter assays (e.g., fluorescent proteins, colorimetric assays, luciferase), DNA sequencing, and/or PCR-based reactions.

Sensors comprising multiple output molecules are also contemplated. In some embodiments, the sensor circuit comprises two output proteins. In some embodiments, the first output molecule and/or the second output molecule is a therapeutic molecule. In some embodiments, the first output molecule and the second output molecule are the same molecule. In some embodiments, the therapeutic molecule is an anti-inflammatory molecule. In some embodiments, the anti-inflammatory molecule is a cytokine, optionally IL-10. In some embodiments, the anti-inflammatory molecule is curcumin. In some embodiments, the anti-inflammatory molecule is an antibody or antibody fragment. Other non-limiting examples of therapeutic molecules contemplated herein include antibodies, single variable domains, scFv-fragments, 5-aminosalicylates, corticosteroids, immunosuppressive agents, antibiotics, and RNAi molecules or guideRNA molecules targeting inflammatory pathways, antibodies/antibody fragments against interleukins or communication molecules themselves (such as TNF-alpha or IL-16), as well as antibodies/antibody fragments against communication molecule receptors/signal-processing pathways. Additionally, anti-inflammatory agonists that turn on anti-inflammatory pathways.

Components (for example, promoters, ribosome binding sites and/or output molecules) of biological sensor circuits may be on a vector. In some embodiments, the promoters are on the same vector (e.g., plasmid). In some embodiments, the promoters are on different vectors (e.g., each on a separate plasmid). In some embodiments, promoters may be on the same vector high copy plasmid, medium copy plasmid, or low copy plasmid. In some embodiments, output molecule(s) of biological analog signal processing circuits may be on a bacterial artificial chromosome (BAC). In some embodiments, sensor circuits are integrated into the genome of an organism.

### Methods of Diagnosing and Treating IBD

The present disclosure is based upon the surprising discovery that probiotics comprising sensor circuits can be consumed orally in order to diagnose and treat IBD as they transit through the gut. Furthermore, engineered probiotics can be recovered from stool and interrogated to recover information on the conditions they encountered during their transit through the gut. Accordingly, the disclosure provides methods of diagnosing and/or treating an inflammatory bowel disease in a subject in need thereof. In some embodiments, the inflammatory bowel disease is Crohn's disease. In some embodiments, the inflammatory bowel disease is ulcerative colitis.

In some aspects, the disclosure relates to a method of treating an inflammatory bowel disease in a subject in need thereof, the method comprising administering to a subject in need thereof a probiotic cell comprising a sensor circuit as described herein. Administering the pharmaceutical composition of the present disclosure may be accomplished by any means known to the skilled artisan. Routes of administration include but are not limited to oral, parenteral, intravenous, intramuscular, intraperitoneal, intranasal, sublingual, intratracheal, inhalation, subcutaneous, ocular, vaginal, and rectal.

In some embodiments, the probiotic cell is administered orally. For oral administration, the compounds can be formulated readily by combining the cell(s) with pharmaceutically acceptable carriers well known in the art. Such carriers enable the compounds of the present disclosure to be formulated as tablets, pills, dragees, capsules, liquids, gels, syrups, slurries, suspensions and the like, for oral ingestion by a subject to be treated. In some embodiments, the probiotic cell is administered as part of a probiotic formulation, optionally as a component in a food product.

As used herein, the term “subject in need thereof” refers to any animal that has signs or symptoms associated with, or is suspected of having, an inflammatory bowel disease. In some embodiments, the subject is a mammal. In some embodiments, the subject is a human, non-human primate, equine, porcine, canine, or feline subject.

In some embodiments of the method, the sensor circuit is a biological analog signal processing circuit. In some embodiments of the method, the input signal is hydrogen peroxide (H2O2). In some embodiments of the method, the input signal is nitric oxide (NO). In some embodiments of the method, the input molecule is an inflammatory cytokine, optionally IL-6, IL-18, or TNFα. In some embodiments, the input molecule is a molecule produced by neutrophils, such as calprotectin or lactoferrin. In some embodiments, the input signal is blood

In some embodiments of the method, the first and/or second output molecule is detectable. In some embodiments of the method, the first and/or second output molecule is detectable by PCR, DNA sequencing, colorimetric observation, or microscopy, optionally fluorescent microscopy.

In some embodiments of the method, the first and/or second output molecule is a therapeutic molecule. In some embodiments of the method, the therapeutic molecule is an anti-inflammatory molecule. In some embodiments of the method, the anti-inflammatory molecule is a cytokine, optionally IL-10. In some embodiments of the method, the anti-inflammatory molecule is curcumin. In some embodiments of the method, the anti-inflammatory molecule is an antibody or antibody fragment. In some embodiments of the method, the sensor circuit further comprises a second output molecule. In some embodiments of the method, the second output molecule is the same as the first output molecule.

Some aspects of the disclosure relate to a method of diagnosing an inflammatory bowel disease in a subject, the method comprising: a) administering to a subject a probiotic cell as described herein; b) obtaining a biological sample from the subject of (a); c) detecting the expression of the at least one output molecule in the biological sample; and, d) diagnosing the subject as having an inflammatory bowel disease or a flare-up of inflammatory bowel diseases.

In some embodiments of the method, the probiotic cell colonizes the intestinal tract. In some embodiments of the method, the subject is a mammal. In some embodiments of the method, the subject is a human.

As used herein, the term “biological sample” refers to a specimen obtained from a subject from which inflammatory markers can be identified. Examples of biological samples include but are not limited to tissue, blood, saliva, urine and fecal samples. In some embodiments of the method, the biological sample is a fecal sample.

In some embodiments of the method, the expression of the output molecule is detected in vitro. In some embodiments of the method, the inflammatory bowel disease is Crohn's disease or ulcerative colitis. In some embodiments, the method further comprises administering an agent useful for treatment of IBD to the subject.

The present disclosure also provides aspects encompassed by the following numbered paragraphs:

1. A recombinant probiotic cell comprising: a sensor circuit, comprising: (a) a first promoter operably linked to a nucleic acid encoding a regulatory protein responsive to an input signal; (b) a second promoter responsive to the regulatory protein and operably linked to a nucleic acid encoding a first output protein, wherein activity of the second promoter is altered when bound by the regulatory protein; (c) an output molecule operably linked to a third promoter, wherein the output molecule or the third promoter is flanked by a first set of regulatory sequences, wherein the first set of regulatory sequences interacts with the first output protein to unlink the output molecule from the third promoter; (d) a fourth promoter responsive to the regulatory protein and operably linked to a nucleic acid encoding a second output protein, wherein activity of the fourth promoter is altered when bound by the regulatory protein; and, optionally, (e) a second output molecule flanked by a second set of regulatory sequences, wherein the second set of regulatory sequences interacts with the second output protein to operably link the second output molecule to a fifth promoter.

2. The recombinant probiotic cell of paragraph 1, wherein the promoter of (a) is a constitutively-active promoter.

3. The recombinant probiotic cell of paragraph 1 or 2, wherein the regulatory protein is selected from the group consisting of oxyR, NorR, and NsrR.

4. The recombinant probiotic cell of any one of paragraphs 1 to 3, wherein the input signal is hydrogen peroxide (H₂O₂).

5 The recombinant probiotic cell of any one of paragraphs 1 to 3, wherein the input signal is nitric oxide (NO).

6. The recombinant probiotic cell of paragraph 1 or 2, wherein the input signal is an inflammatory cytokine, optionally IL-6, IL-18, or TNFα.

7. The recombinant probiotic cell of any one of paragraphs 1 to 6, wherein the promoter of (b) and/or (d) comprises a modification that alters the binding affinity of a transcription factor or RNA polymerase for the promoter of (b) and/or (d), relative to a similar unmodified promoter.

8. The recombinant probiotic cell of paragraph 7, wherein the modification is a nucleic acid mutation.

9. The recombinant probiotic cell of any one of paragraphs 1 to 8, wherein (a), (b) and (c) are on a vector.

10. The recombinant probiotic cell of paragraph 9, wherein (a), (b), (c) and (d) are on a vector.

11. The recombinant probiotic cell of any one of paragraphs 1 to 10, wherein (a) and (b) are on a single vector.

12. The recombinant probiotic cell of paragraph 11, wherein (a), (b) and (d) are on a single vector.

13. The recombinant probiotic cell of any one of paragraphs 1 to 12, wherein (c) and/or (e) is on a bacterial artificial chromosome (BAC).

14. The recombinant probiotic cell of any one of paragraphs 1 to 13, wherein (b) and/or (d) further comprises a sequence element that regulates production of the first output protein and is located between the second promoter and the nucleic acid encoding the first output protein.

15. The recombinant probiotic cell of paragraph 14, wherein the sequence element regulates transcription or translation of the output protein.

16. The recombinant probiotic cell of paragraph 14 or paragraph 15, wherein the sequence element is a ribosomal binding site.

17. The recombinant probiotic cell of paragraph 16, wherein the sequence element is a modified ribosomal binding site comprising a modification that alters the binding affinity of a ribosome for the modified ribosomal binding site, relative to a similar unmodified ribosomal binding site.

18. The recombinant probiotic cell of any one of paragraphs 1 to 17, wherein the promoter of (b) and/or (d) is a promoter selected from the group consisting of oxyR, oxySp, katGp, nir, hcp, nrfA, nasD, ytfE, yeaR, nnrS and norV that comprises a modification that alters the binding affinity of a transcription factor or RNA polymerase for a promoter selected from the group consisting of oxyR, nir, hcp, nrfA, nasD, ytfE, yeaR, nnrS and norV of (b), relative to a similar unmodified promoter.

19. The recombinant probiotic cell of any one of paragraphs 1 to 17, wherein the promoter of (b) and/or (d) is a promoter selected from the group consisting of oxyR, oxySp, katGp, nir, hcp, nrfA, nasD, ytfE, yeaR, nnrS and norV that is a naturally occurring promoter.

20. The recombinant probiotic cell of any one of paragraphs 1 to 19, wherein the first output protein of (b) is a recombinase and the first set of regulatory sequences of (c) is recombinase recognition sites.

21. The recombinant probiotic cell of any one of paragraphs 1 to 20, wherein the second output protein of (d) is a recombinase and the second set of regulatory sequences of (e) is recombinase recognition sites.

22. The recombinant probiotic cell of any one of paragraphs 1 to 21, wherein the first output molecule and/or the second output molecule is detectable.

23. The recombinant probiotic cell of paragraph 22, wherein the first output molecule and/or the second output molecule is detectable by PCR, DNA sequencing or microscopy, optionally fluorescent microscopy.

24. The recombinant probiotic cell of any one of paragraphs 1 to 23, wherein the first output molecule and/or the second output molecule is a therapeutic molecule.

25. The recombinant probiotic cell of any one of paragraphs 1 to 24, wherein the first output molecule and the second output molecule are the same molecule.

26. The recombinant probiotic cell of paragraph 24, wherein the therapeutic molecule is an anti-inflammatory molecule.

27. The recombinant probiotic cell of paragraph 26, wherein the anti-inflammatory molecule is a cytokine, optionally IL-10.

28. The recombinant probiotic cell of paragraph 26, wherein the anti-inflammatory molecule is curcuminoid synthase (CUS) that converts feruloyl-CoA to curcumin.

29. The recombinant probiotic cell of any one of paragraphs 1 to 28, wherein the cell is a bacterial cell or a fungal cell.

30. The recombinant probiotic cell of paragraph 29, wherein the bacterial cell is an E. coli cell, optionally an E. coli Nissle 1917 cell.

31. The recombinant probiotic cell of paragraph 29, wherein the fungal cell is a yeast cell, optionally, a Saccharomyces boulardii cell.

32. A method of treating an inflammatory bowel disease in a subject in need thereof, the method comprising administering to a subject in need thereof the probiotic cell of any one of paragraphs 1 to 31.

33. The method of paragraph 32, wherein the inflammatory bowel disease is Crohn's disease or ulcerative colitis.

34. The method of paragraph 32 or 33, wherein the sensor circuit is a biological analog signal processing circuit.

35. The method of any one of paragraphs 32 to 34, wherein the input signal is hydrogen peroxide (H₂O₂).

36. The method of any one of paragraphs 32 to 34, wherein the input signal is nitric oxide (NO).

37 The method of any one of paragraphs 32 to 34, wherein the input molecule is an inflammatory cytokine, optionally IL-6, IL-18, or TNFα.

38. The method of any one of paragraphs 32 to 37, wherein the output molecule is detectable.

39. The method of any one of paragraphs 32 to 38, wherein the output molecule is detectable by PCR, DNA sequencing or microscopy, optionally fluorescent microscopy.

40. The method of any one of paragraphs 32 to 39, wherein the output molecule is a therapeutic molecule.

41. The method of paragraph 40, wherein the therapeutic molecule is an anti-inflammatory molecule.

42. The method of paragraph 41, wherein the anti-inflammatory molecule is a cytokine, optionally IL-10.

43. The method of paragraph 41, wherein the anti-inflammatory molecule is curcuminoid synthase (CUS) that converts feruloyl-CoA to curcumin.

44. The method of any one of paragraphs 32 to 43, wherein the sensor circuit further comprises a second output molecule.

45. The method of any one of paragraphs 32 to 44, wherein the second output molecule is the same as the first output molecule.

46. A method of diagnosing an inflammatory bowel disease in a subject, the method comprising: (a) administering to a subject the probiotic cell of any one of paragraphs 1 to 16; (b) obtaining a biological sample from the subject of (a); (c) detecting the expression of the at least one output molecule in the biological sample; and (d) diagnosing the subject as having an inflammatory bowel disease.

47. The method of paragraph 46, wherein the probiotic cell colonizes the intestinal tract.

48. The method of paragraph 46 or 47, wherein the subject is a mammal.

49. The method of paragraph 48, wherein the subject is a human.

50. The method of any one of paragraphs 46 to 49, wherein the biological sample is a fecal sample.

51. The method of any one of paragraphs 46 to 50, wherein the expression of the output molecule is detected in vitro.

52. The method of any one of paragraphs 46 to 51, wherein the inflammatory bowel disease is Crohn's disease or ulcerative colitis.

53. The method of any one of paragraphs 46 to 52, wherein the method further comprises (e) administering an agent useful for treatment of IBD to the subject.

54. A recombinant probiotic cell comprising: a sensor circuit, comprising: (a) a first promoter operably linked to a nucleic acid encoding a regulatory protein responsive to an input signal; and (b) a second promoter responsive to the regulatory protein and operably linked to a nucleic acid encoding a first output protein, wherein activity of the second promoter is altered when bound by the regulatory protein.

55. The recombinant probiotic cell of paragraph 1, wherein the promoter of (a) is a constitutively-active promoter.

56. The recombinant probiotic cell of paragraph 54 or 55, wherein the regulatory protein is selected from the group consisting of oxyR, NorR, and NsrR.

57. The recombinant probiotic cell of any one of paragraphs 54 to 56, wherein the input signal is hydrogen peroxide (H₂O₂).

58. The recombinant probiotic cell of any one of paragraphs 54 to 56, wherein the input signal is nitric oxide (NO).

59. The recombinant probiotic cell of paragraph 54 or 55, wherein the input signal is an inflammatory cytokine, optionally IL-6, IL-18, or TNFα.

60. The recombinant probiotic cell of any one of paragraphs 54 to 59, wherein the promoter of (b) comprises a modification that alters the binding affinity of a transcription factor or RNA polymerase for the promoter of (b), relative to a similar unmodified promoter.

61. The recombinant probiotic cell of paragraph 60, wherein the modification is a nucleic acid mutation.

62. The recombinant probiotic cell of any one of paragraphs 54 to 61, wherein (a) and (b) are on a vector.

63. The recombinant probiotic cell of any one of paragraphs 54 to 62, wherein (a) and (b) are on a single vector.

64. The recombinant probiotic cell of any one of paragraphs 54 to 63, wherein (b) further comprises a sequence element that regulates production of the first output protein and is located between the second promoter and the nucleic acid encoding the first output protein.

65. The recombinant probiotic cell of paragraph 64, wherein the sequence element regulates transcription or translation of the output protein.

66. The recombinant probiotic cell of paragraph 64 or paragraph 65, wherein the sequence element is a ribosomal binding site.

67. The recombinant probiotic cell of paragraph 66, wherein the sequence element is a modified ribosomal binding site comprising a modification that alters the binding affinity of a ribosome for the modified ribosomal binding site, relative to a similar unmodified ribosomal binding site.

68. The recombinant probiotic cell of any one of paragraphs 54 to 67, wherein the promoter of (b) is a promoter selected from the group consisting of oxyR, oxySp, katGp, nir, hcp, nrfA, nasD, ytfE, yeaR, nnrS and norV that comprises a modification that alters the binding affinity of a transcription factor or RNA polymerase for a promoter selected from the group consisting of oxyR, nir, hcp, nrfA, nasD, ytfE, yeaR, nnrS and norV of (b), relative to a similar unmodified promoter.

69. The recombinant probiotic cell of any one of paragraphs 54 to 68, wherein the promoter of (b) is a promoter selected from the group consisting of oxyR, oxySp, katGp, nir, hcp, nrfA, nasD, ytfE, yeaR, nnrS and norV that is a naturally occurring promoter.

70. The recombinant probiotic cell of any one of paragraphs 54 to 69, wherein the first output molecule is a therapeutic molecule.

71. The recombinant probiotic cell of paragraph 70, wherein the therapeutic molecule is an anti-inflammatory molecule.

72. The recombinant probiotic cell of paragraph 71, wherein the anti-inflammatory molecule is a cytokine, optionally IL-10.

73. The recombinant probiotic cell of paragraph 71, wherein the anti-inflammatory molecule is curcuminoid synthase (CUS) that converts feruloyl-CoA to curcumin.

74. The recombinant probiotic cell of paragraph 71, further comprising nucleic acids that encode 4-coumarate:CoA ligase and acetyl-CoA carboxylase, which nucleic acids optionally are on one or more vectors.

75. The recombinant probiotic cell of paragraph 74, wherein the nucleic acids that encode acetyl-CoA carboxylase are AccBc and DtsR1.

76. The recombinant probiotic cell of paragraph 74 or 75, wherein nucleic acids that encode 4-coumarate:CoA ligase and acetyl-CoA carboxylase are operably linked to constitutive promoters.

77. The recombinant probiotic cell of any one of paragraphs 54 to 76, wherein the cell is a bacterial cell or a fungal cell.

78. The recombinant probiotic cell of paragraph 77, wherein the bacterial cell is an E. coli cell, optionally an E. coli Nissle 1917 cell.

79. The recombinant probiotic cell of claim 77, wherein the fungal cell is a yeast cell, optionally, a Saccharomyces boulardii cell.

80. A method of treating an inflammatory bowel disease in a subject in need thereof, the method comprising administering to a subject in need thereof the probiotic cell of any one of claims 54 to 79.

81. The method of claim 80, wherein the inflammatory bowel disease is Crohn's disease or ulcerative colitis.

82. A recombinant cell comprising:

a sensor circuit, comprising: (a) a first promoter operably linked to a nucleic acid encoding a regulatory protein responsive to an input signal; (b) a second promoter responsive to the regulatory protein and operably linked to a nucleic acid encoding a first output protein, wherein the second promoter is a nir, hcp, nrfA, nasD, ytfE, yeaR, nnrS or norV promoter, and wherein activity of the second promoter is altered when bound by the regulatory protein; and (c) an output molecule flanked by a first set of regulatory sequences, wherein the first set of regulatory sequences interacts with the first output protein to operably link the output molecule to a third promoter.

83. The recombinant cell of paragraph 82 further comprising: (d) a fourth promoter responsive to the regulatory protein and operably linked to a nucleic acid encoding a second output protein, wherein activity of the fourth promoter is altered when bound by the regulatory protein.

84. The recombinant cell of paragraph 83 further comprising: (e) a second output molecule flanked by a second set of regulatory sequences, wherein the second set of regulatory sequences interacts with the second output protein to operably link the second output molecule to a fifth promoter.

85. The recombinant cell of any one of paragraphs 82-84, wherein the regulatory protein is NsrR.

86. The recombinant cell of any one of paragraphs 82-84, wherein the regulatory protein is NorR.

87. The recombinant cell of any one of paragraphs 82-86, wherein the input signal is nitric oxide (NO).

88. The recombinant cell of paragraph 82, wherein the input signal is an inflammatory cytokine, optionally IL-6, IL-18, or TNFα.

89. The recombinant cell of any one of paragraphs 82-88, wherein the first promoter of (a) is a constitutively-active promoter.

90. The recombinant cell of any one of paragraphs 82-89, wherein the second promoter of (b) comprises a modification that alters the binding affinity of a transcription factor or RNA polymerase for the second promoter of (b), relative to a similar unmodified promoter.

91. The recombinant cell of paragraph 90, wherein the modification is a nucleic acid mutation.

92. The recombinant cell of any one of paragraphs 82-91, wherein (a) and (b) are on a single vector.

93. The recombinant cell of any one of paragraphs 82-92, wherein (b) further comprises a sequence element that regulates production of the first output protein and is located between the second promoter and the nucleic acid encoding the first output protein.

94. The recombinant cell of paragraph 93, wherein the sequence element regulates transcription or translation of the output protein.

95. The recombinant cell of paragraph 93 or 94, wherein the sequence element is a ribosomal binding site.

96. The recombinant cell of paragraph 94 or 95, wherein the sequence element is a modified ribosomal binding site comprising a modification that alters the binding affinity of a ribosome for the modified ribosomal binding site, relative to a similar unmodified ribosomal binding site.

97. The recombinant cell of any one of paragraphs 82-96, wherein the first output protein of (b) is a recombinase and the first set of regulatory sequences of (c) comprises recombinase recognition sites.

98. The recombinant cell of any one of paragraphs 82-97, wherein the first output protein or second output protein is detectable.

99. The recombinant cell of any one of paragraphs 82-98, wherein the first output protein or second output protein is detectable by PCR, DNA sequencing or microscopy, optionally fluorescent microscopy.

100. The recombinant cell of any one of paragraphs 82-99, wherein the output molecule of (c) is a therapeutic molecule.

101. The recombinant cell of paragraph 100, wherein the therapeutic molecule is an anti-inflammatory molecule.

102. The recombinant cell of paragraph 101, wherein the anti-inflammatory molecule is a cytokine.

103. The recombinant cell of paragraph 102, wherein the cytokine is IL-10.

104. The recombinant cell of paragraph 101, wherein the anti-inflammatory molecule is curcuminoid synthase (CUS).

105. The recombinant cell of any one of paragraphs 82-104, wherein the cell is a bacterial cell or a fungal cell.

106. The recombinant cell of paragraph 105, wherein the cell is an Escherichia coli cell

107. The recombinant cell of paragraph 105, wherein the cell is a Saccharomyces boulardii cell.

108. The recombinant cell of any one of paragraphs 82-107, wherein the recombinant cell is a recombinant probiotic cell.

109. A method of treating an inflammatory bowel disease in a subject in need of treatment of an inflammatory bowel disease, the method comprising administering to a subject having an inflammatory bowel disease the cell of any one of paragraphs 82-108.

110. The method of paragraph 109, wherein the inflammatory bowel disease is Crohn's disease or ulcerative colitis.

### EXAMPLES

### Introduction

The present disclosure is related, in part, to the engineering of a suite of probiotic bacteria as in vivo sensors for inflammation and localized therapeutics. Although many powerful synthetic gene circuits have been described in the last decade, few have been applied to study and manipulate human diseases. The probiotic cells described herein are useful for studying inflammation in both healthy and diseased environments, allowing for the identification of the timing and concentration of key molecules that initiate and contribute to the development of IBD and the design of diagnostics for early detection of IBD flares. Precise in vivo profiles for inflammatory mediators in IBD have not been mapped out, even though they are essential to understand for the development of more effective therapeutics. As described herein, probiotic cells comprising diagnostic sensors are also engineered to express anti-inflammatory therapeutics on-demand, thus resulting in intelligent drugs that make decisions about the timing, dosage, and location of IBD therapeutics. Prior work on probiotics has utilized constitutive production of anti-inflammatory drugs, but these have not shown good efficacy in clinical trials.

### Example 1: Probiotic Sensors and Memory Circuits for Inflammation

In this example, probiotic bacteria, such as E. coli Nissle 1917, are engineered to detect and remember the presence and concentration of inflammatory mediators, such as H₂O₂ and NO, via sensor circuits (FIG. 2). Specifically, the probiotic cells are engineered to comprise a suite of orthogonal recombinases that are expressed under multiple independent circuits and that are induced by different levels of inflammation. As illustrated in FIG. 2, the expression of Recombinase 1 is induced when the inflammation sensor exceeds a low threshold, the expression of Recombinase 2 is induced when the inflammation sensor exceeds a medium threshold, and expression of Recombinase 3 is induced when the inflammation sensor exceeds a high threshold.

When expressed, each of these recombinases flips a specific stretch of DNA, thus creating a stable memory of events that is read out via reporter assays (e.g., fluorescent proteins, colorimetric assays, luciferase), DNA sequencing, and/or PCR-based reactions (3).

### Results

Engineered bacterial cells containing two orthogonal sensors for detecting H₂O₂ and paraquat (a superoxide generator) were engineered. The results demonstrate that the engineered bacteria can distinguish between wild-type mammalian immune cells and those with an IBD-relevant mutation (4). Furthermore, two E. coli strains that can sense different concentrations of H₂O₂ by using the OxyR transcription factor have been produced. OxyR is normally in a reduced form, but once it reacts with H₂O₂, it is converted into its oxidized form, which binds to specific DNA regulatory elements in targeted promoters (e.g. ahpCp, katGp, oxyRp, oxySp) (7-9).

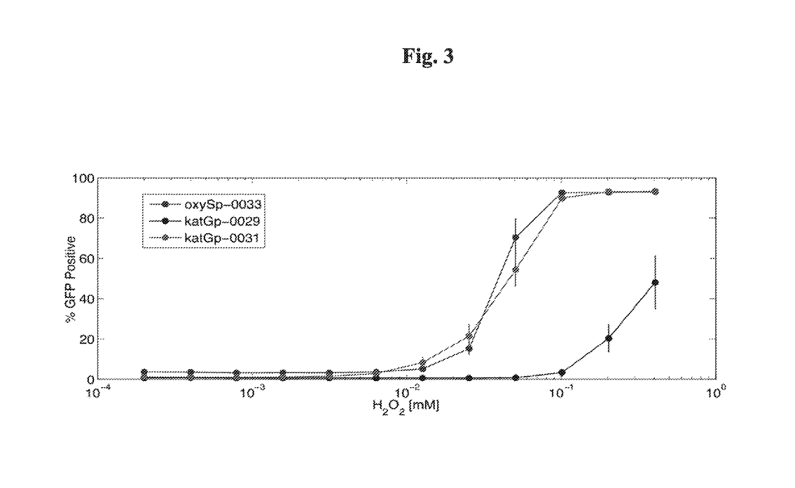

Six combinatorial designs for H₂O₂ sensing were produced by assembling two promoters (katGp and oxySp) with three ribosome-binding sites (BBa_0029, BBa_0031, BBa_0033 from the Registry of Standard Biological Parts) to control the expression of GFP, thus modulating both transcription and translation. Three of these constructs had constitutively high activity irrespective of the H₂O₂ concentration. The remaining three (shown in FIG. 3; oxySp+0033, katGp+0031, katGp+0029) exhibited two distinct thresholds in the transfer functions between H₂O₂ (input) and the % GFP-positive cells (output) (FIG. 3). These results demonstrate genetic circuits in which different sensed concentrations of H₂O₂ induce different gene expression profiles, thus enabling the sensing of various levels of inflammation.

### Development of Nitric Oxide Sensor

NO-sensitive transcription factors, for example NorR (15) and NsrR (16), are combined with a variety of their respective promoters, including the nir (16), hcp, nrfA (18), nasD (19), ytfE (18, 20), yeaR, nnrS (20) and norV (21) promoters to engineer a class of NO sensor circuits in probiotic cells. To screen for functional sensor circuits, NsrR and NorR are placed under the control of a IPTG-inducible promoter (pLlacO) on a low copy plasmid. The NO-sensitive promoters control a Flavin-based reporter gene (e.g. EcFpFB or iLOV) (22), that does not require O₂ for maturation, as an output molecule on the same plasmid. NO sensor circuits are transformed into E. coli MG1655-Pro, a strain that expresses Lad from the genome, by inducing with increasing concentrations of IPTG and NO-generating molecules, such as sodium nitroprusside or diethylenetriamine/nitric oxide. The NO sensing circuit is endogenously designed to function anaerobically or micro-aerobically.

### Example 2: Design Probiotic Bacteria for Localized Sensing-and-Treatment of IBD

Probiotics that produce and secrete anti-inflammatory compounds in response to inflammation offer a solution to the challenge of orally delivering peptides and some small molecules for IBD treatment. This example describes the construction of intelligent probiotics that release anti-inflammatory compounds only if, where, and when they are needed, thereby potentially reducing the side effects currently associated with IBD therapy and increasing success rates by treating IBD flare-ups prior to clinical presentation. Intelligent therapeutics must be rapidly released after detecting inflammation to ensure that they reach the correct site in a timely fashion.

Engineered probiotic cells with inflammation sensor circuits expressing anti-inflammatory protein and/or anti-inflammatory small molecules are constructed. Non-limiting examples of useful output molecules include the cytokine IL-10, anti-TNFα antibodies, antibody fragments, and the small molecule curcumin. Sensor circuit designs are inserted into probiotic bacteria and tested in vitro by inducing with various concentrations of reactive oxygen species and measuring the time response and the titers of the resulting anti-inflammatory compounds.

Mutant strains of E. coli are needed for the production of cytokines and antibodies. Some eukaryotic proteins require disulfide bond formation; however, it is challenging to fold these molecules correctly in the naturally oxidizing cytoplasm of E. coli. Therefore, a previously established mutant E. coli strain with a reducing cytoplasm (10) is used to express active anti-inflammatory compounds in large quantities. To achieve secretion of active therapeutic molecules into the supernatant, therapeutic proteins are fused to a signaling peptide that uses the well-understood type I secretion system (11). Disulfide bond formation in secreted proteins is confirmed by reducing and non-reducing protein gel electrophoresis. Activity is tested by incubating supernatant with macrophages followed by Western blot analysis on specific transmembrane receptors of the macrophages.

For small-molecule anti-inflammatories, probiotic cells with sensor circuits controlling the expression of curcumin are constructed. Curcumin is a hydrophobic molecule naturally produced by Curcuma longa. It has been shown that curcumin exhibits anti-inflammatory properties, potentially through inhibition of NFκB (12). Curcumin is safe in high doses in humans, but poor bioavailability caused by poor absorption, rapid metabolism, and/or systemic elimination is currently the limiting factor for curcumin as an effective therapeutic (13).

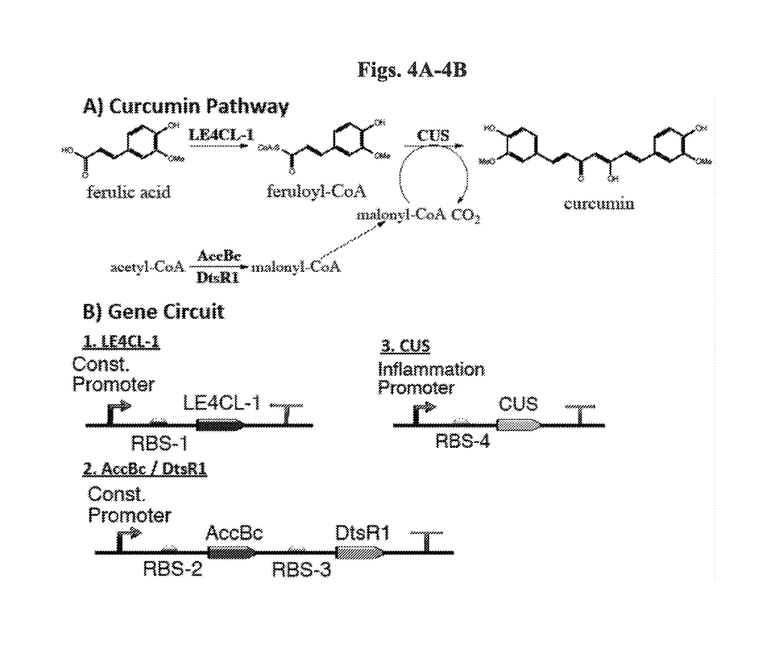

To overcome these issues, probiotic bacteria engineered to sense inflammatory markers and express curcumin in vivo are constructed. A synthetic production pathway to enable the generation of curcumin in E. coli from ferulic acid, a cheap and commercially available compound, has been previously described (14). Ferulic acid is converted to feruloyl-CoA by LE4CL-1 (4-coumarate:CoA ligase (4CL) from Lithospermum erythrorhizon), which in turn, gets converted by curcuminoid synthase (CUS) to curcumin (FIG. 4A). The last step of biosynthesis requires malonyl-CoA as a cofactor. AccBc and DtsR1, two enzymes that form an acetyl-CoA carboxylase complex that transforms acetyl-CoA to malonyl-CoA, are also important. To enable controlled production of curcumin by inflammation-sensing circuits, expression of the curcuminoid synthase (CUS) enzyme, needed for the last conversion step (feruloyl-CoA to curcumin), is placed under regulation by an inflammation sensor.

### REFERENCES

[1] R. Daniel, J. R. Rubens, R. Sarpeshkar, T. K. Lu, Synthetic analog computation in living cells. Nature, 497 (2013), 619.
[2] P. Siuti, J. Yazbek, T. K. Lu, Synthetic circuits integrating logic and memory in living cells. Nat. Biotech, 31 (2013), 448.
[3] P. Siuti, J. Yazbek, T. K. Lu, Engineering genetic circuits that compute and remember. Nature Protoc., 9 (2014), 1292.
[4] J. R. Rubens, I. E. Müller, D. Graham, T. Jun, R. Xavier, T. K. Lu, Gene Circuits that Correct Crosstalk with Crosstalk. Manuscript in preparation
[5] S. Y. Oh, K.-A. Cho, J. L. Kang, K. H. Kim, S.-Y. Woo, Comparison of experimental mouse models of inflammatory bowel disease. International Journal of Molecular Medicine, 33 (2014), 333.
[6] J. R. Maxwell, J. L. Viney, Overview of Mouse Models of Inflammatory Bowel Disease and Their Use in Drug Discovery. Current Protocols in Pharmacology, 5.57.1 (2009).
[7] F. Åslung, M. Zheng, J. Beckwith, G. Storz, Regulation of the OxyR transcription factor by hydrogen peroxide and the cellular thiol-disulfide status. Proc. Natl. Acad. Sci., 96 (1999), 6161.
[8] M. F. Christman, G. Storz, B. N. Ames, OxyR, a positive regulator of hydrogen peroxide-inducible genes in Escherichia coli and Salmonella typhimurium, is homologous to a family of bacteria regulatory proteins. PNAS, 86 (1989), 3484.
[9] S. O. Kim, K. Merchant, R. Nudelman, W. F. Beyer, Jr., T. Keng, J. DeAngelo, A. Hausladen, J. S. Stamler, OxyR: A Molecular Code for Redox-Related Signaling. Cell, 109 (2002), 383.
[10] J. Lobstein, C. A. Emrich, C. Jeans, M. Faulkner, P. Riggs, M. Berkmen, SHuffle, a novel Escherichia coli protein expression strain capable of correctly folding disulfide bonded proteins in its cytoplasm. Microb. Cell Fact., 11 (2012), 56.
[11] P. Delepelaire, Type I secretion in gram-negative bacteria. Biochimica et Biophysica Acta, 1694 (2004), 149.
[12] A. Lubbad, M. A. Oriowo, I. Khan, Curcumin attenuates inflammation through inhibition of TLR-4 receptor in experimental colitis. Mol. Cell Biochem., 322 (2009), 127.
[13] P. Anand, A. B. Kunnumakkara, R. A. Newman, B. B. Aggarwal, Bioavailability of Curcumin: Problems and Promises. Molecular Pharmaceutics, 4 (2007), 807.
[14] Y. Katsuyama, M. Matsuzama, N. Funa, S. Horinouchi, Production of curcuminoids by Escherichia coli carrying an artificial biosynthesis pathway. Microbiology, 154 (2008), 2620.
[15] M. Bush, T. Ghosh, N. Tucker, X. Zhang, R. Dixon, Transcriptional regulation by the dedicated nitric oxide sensor, NorR: a route towards NO detoxification. Biochem. Soc. Trans., 39 (2011), 289.
[16] H. J. E. Beaumont, S. I. Lens, W. N. M. Reijnders, H. V. Westerhoff, R. J. M. van Spanning, Expression of nitrite reductase in Nitrosomonas europaea involves NsrR, a novel nitrite-sensitive transcription repressor. Mol. Microbiol., 54 (2004), 148.
[17] D. M. Bodenmiller, S. Spiro, The yjeB (nsrR) gene of Escherichia coli encodes a nitric oxide-sensitive transcriptional regulator. J. Bacteriol., 188 (2006), 874.
[18] N. Filenko, S. Spiro, D. F. Browning, D. Squire, T. W. Overton, J. Cole, C. Constantinidou, The NsrR Regulon of Escherichia coli K-12 Includes Genes Encoding the Hybrid Cluster Protein and the Periplasmic, Respiratory Nitrite Reductase. J. Bacteriol., 189 (2007), 4410.
[19] S. Kommineni, A. Lama, B. Popescu, M. M. Nakano, Global Transcriptional Control by NsrR in Bacillus subtilis. J. Bacteriol., 194 (2012), 194.
[20] H.-Y. Lin, P. J. Bledsoe, V. Stewart, Activation of yeaR-yoaG Operon Transcription by the Nitrate-Responsive Regulator NarL Is Independent of Oxygen-Responsive Regulator Fnr in Escherichia coli K-12. J. Bacteriol., 189 (2007), 7539.
[21] A. M. Gardner, C. R. Gessner, P. R. Gardner, Regulation of the Nitric Oxide Reduction Operon (noRVW) in Escherichia coli. J. Biol. Chem., 278 (2003), 10081.
[22] A. Mukherjee, J. Walker, K. B. Weyant, C. M. Schroeder, Characterization of Flavin-Based Fluorescent Proteins: An Emerging Class of Fluorescent Reporters. PLoS ONE, 8 (2013), e64753.

1-110. (canceled)

111. A recombinant probiotic cell comprising:
a sensor circuit, comprising:
(a) a first promoter operably linked to a nucleic acid encoding a regulatory protein responsive to an input signal; and
(b) a second promoter responsive to the regulatory protein and operably linked to a nucleic acid encoding a first output protein, wherein activity of the second promoter is altered when bound by the regulatory protein.

112. The recombinant probiotic cell of claim 111, further comprising:
(c) an output molecule flanked by a first set of regulatory sequences, wherein the first set of regulatory sequences interacts with the first output protein to operably link the output molecule to a third promoter.

113. The recombinant probiotic cell of claim 112, further comprising:
(d) a fourth promoter responsive to the regulatory protein and operably linked to a nucleic acid encoding a second output protein, wherein activity of the fourth promoter is altered when bound by the regulatory protein.

114. The recombinant probiotic cell of claim 113, further comprising:
(e) a second output molecule flanked by a second set of regulatory sequences, wherein the second set of regulatory sequences interacts with the second output protein to operably link the second output molecule to a fifth promoter.

115. A recombinant probiotic cell comprising:
a sensor circuit, comprising:
(a) a first promoter operably linked to a nucleic acid encoding a regulatory protein responsive to an input signal;
(b) a second promoter responsive to the regulatory protein and operably linked to a nucleic acid encoding a first output protein, wherein activity of the second promoter is altered when bound by the regulatory protein;
(c) an output molecule operably linked to a third promoter, wherein the output molecule or the third promoter is flanked by a first set of regulatory sequences, wherein the first set of regulatory sequences interacts with the first output protein to unlink the output molecule from the third promoter;
(d) a fourth promoter responsive to the regulatory protein and operably linked to a nucleic acid encoding a second output protein, wherein activity of the fourth promoter is altered when bound by the regulatory protein; and, optionally,
(e) a second output molecule flanked by a second set of regulatory sequences, wherein the second set of regulatory sequences interacts with the second output protein to operably link the second output molecule to a fifth promoter.

116. The recombinant probiotic cell of claim 111, wherein the promoter of (a) is a constitutively-active promoter.

117. The recombinant probiotic cell of claim 111, wherein the regulatory protein is selected from the group consisting of oxyR, NorR, and NsrR.

118. The recombinant probiotic cell of claim 111, wherein the input signal is hydrogen peroxide (H2O2).

119. The recombinant probiotic cell of claim 111, wherein the input signal is nitric oxide (NO).

120. The recombinant probiotic cell of claim 111, wherein the input signal is an inflammatory cytokine, optionally IL-6, IL-18, or TNFα.

121. The recombinant probiotic cell of claim 111, wherein the promoter of (b) and/or (d) is selected from the group consisting of a oxyR, oxySp, katGp, nir, hep, mfA, nasD, ytfE, year, nnrS, and norV promoter.

122. The recombinant probiotic cell of claim 111, wherein the promoter of (b) and/or (d) comprises a modification that alters the binding affinity of a transcription factor or RNA polymerase for the promoter of (b) and/or (d), relative to a similar unmodified promoter.

123. The recombinant probiotic cell of claim 122, wherein the modification is a nucleic acid mutation.

124. The recombinant probiotic cell of claim 111, wherein (b) and/or (d) further comprises a sequence element that regulates production of the first output protein and is located between the second promoter and the nucleic acid encoding the first output protein.

125. The recombinant probiotic cell of claim 124, wherein the sequence element regulates transcription or translation of the output protein.

126. The recombinant probiotic cell of claim 124, wherein the sequence element is a ribosomal binding site.

127. The recombinant probiotic cell of claim 126, wherein the sequence element is a modified ribosomal binding site comprising a modification that alters the binding affinity of a ribosome for the modified ribosomal binding site, relative to a similar unmodified ribosomal binding site.

128. The recombinant probiotic cell of claim 111, wherein the first output molecule and/or the second output molecule is a therapeutic molecule.

129. The recombinant probiotic cell of claim 128, wherein the therapeutic molecule is an anti-inflammatory molecule.

130. The recombinant probiotic cell of claim 129, wherein the anti-inflammatory molecule is a cytokine, optionally IL-10.

131. The recombinant probiotic cell of claim 111, wherein the cell is a bacterial cell or a fungal cell.

132. The recombinant probiotic cell of claim 131, wherein the bacterial cell is an E. coli cell, optionally an Escherichia coli Nissle 1917 cell.

133. The recombinant probiotic cell of claim 131, wherein the fungal cell is a yeast cell, optionally, a Saccharomyces boulardii cell.

134. A method of treating an inflammatory bowel disease in a subject in need thereof, the method comprising administering to a subject in need thereof the probiotic cell of claim 111.

135. The method of claim 134, wherein the inflammatory bowel disease is Crohn's disease or ulcerative colitis.
